(19) United States
(12) Patent Application Publication
(10) Pub. No.: US 2015/0086626 A1
(43) Pub. Date: Mar. 26, 2015

(54) STABLE DOSAGE FORMS OF SKELETAL MUSCLE RELAXANTS WITH EXTENDED RELEASE COATING

(71) Applicant: MYLAN, INC, Morgantown, WV (US)

(72) Inventors: Thomas Daniel Reynolds, Morgantown, WV (US); Boyong Li, Morgantown, WV (US)

(21) Appl. No.: 14/394,666

(22) PCT Filed: Apr. 16, 2013
(86) PCT No.: PCT/US13/36775

§ 371 Date: Oct. 15, 2014

Related U.S. Application Data
(60) Provisional application No. 61/625,600, filed on Apr. 17, 2012.
(60) Provisional application No. 61/635,120, filed on Apr. 18, 2012.

(51) Int. Cl. A61K 9/50; A61K 31/137

(52) U.S. Cl. CPC A61K 9/5047; A61K 31/137
USPC 424/461; 424/494; 514/654; 424/495

(57) ABSTRACT

A unit dosage form, such as a capsule or the like, for delivering a skeletal muscle relaxant, such as cyclobenzaprine hydrochloride, into the body in an extended or sustained release fashion comprising one or more populations of drug-containing particles (beads, pellets, granules, etc.) is disclosed along with a method of preparation therefor. The dosage form comprises active core particles which are individually surrounded by a water insoluble polymer coating which lacks plasticizer.

### CROSS-REFERENCE TO RELATED APPLICATIONS

This application claims the benefit of U.S. Provisional Application No. 61/625,600, filed on Apr. 17, 2012, and U.S. Provisional Application No. 61/635,120, filed on Apr. 18, 2012, the entire disclosures of which are incorporated herein by reference.

### BACKGROUND

1. Field of the Disclosure

This disclosure relates generally to a modified release dosage form of skeletal muscle relaxant comprising skeletal muscle relaxant-containing core particles individually surrounded by an extended release membrane composition comprising a water insoluble polymer, wherein the membrane composition is substantially free of plasticizer.

2. Description of Related Art

U.S. Pat. No. 4,851,228 to Zentner et al. teaches a multiparticulate controlled release dosage form comprising an active agent such as cyclobenzaprine and a polymer coating of various water insoluble polymers and plasticizers. The reference also teaches including pore formers which would alter the release profile (abstract; claims 1, 9 and 16; col. 7, lines 25-63).

U.S. Pat. No. 5,120,548 to McLelland et al. discloses a modified release formulation wherein the active pharmaceutical-containing core is coated with a semi-permeable wall-forming material containing cellulose acetate.

U.S. Pat. No. 6,344,215 to Bettman et al. discloses that dissolution rate controlling polymers, such as ethylcellulose, used in forming membrane coatings are usually plasticized. Plasticizers are added to membrane coatings to enhance coating flexibility, which prevents attrition of the coating, especially during application to a core particle. Representative examples of plasticizers that may be used to plasticize the membranes include triacetin, tributyl citrate, triethyl citrate, acetyl tri-n-butyl citrate, diethyl phthalate, castor oil, dibutyl sebacate, acetylated monoglycerides and the like or mixtures thereof. The plasticizer may comprise about 3 to 30 wt. %, more typically about 10 to 25 wt. %, based on the polymer content of the coating. The plasticizer is selected based on the total solids in the coating system (dissolved or dispersed) and depends on the polymer or polymers and the nature of the coating medium.

U.S. Pat. No. 7,387,793 to Venkatesh et al. teaches a water insoluble polymer based extended release membrane coat. A plasticizer is provided to resist attrition from the coating process and retain the integrity of the membrane during dissolution in an aqueous medium.

Inclusion of a plasticizer normally increases the complexity of the extended release membrane system in terms of plasticizer types, levels and plasticizing time during preparation. The migration of the plasticizer out of membrane can occur during long term storage, causing change of drug release over time or interaction of the plasticizer with the drug. Therefore, a seal coat is typically used to protect the active ingredient-containing core and a curing step of the coated core is carried out to minimize the change of membrane properties over time.

Accordingly, it would be desirable to provide an innovative formulation and process to obtain a water insoluble polymer based extended release membrane coat without using a plasticizer, provided that the membrane prepared without any plasticizer can resist the attrition from a coating process and retain integrity during dissolution in an aqueous medium. The benefits of an extended release membrane without plasticizer include (1) less complexity of the membrane, (2) no complications caused from plasticizer leaching out of the membrane, such as changes in drug release rate and interaction with the drug in the core, and (3) a simpler manufacturing process with no need for curing steps. Moreover, it has been found in certain embodiments of the disclosure that potency of the active pharmaceutical ingredient (% theoretical drug content) can be enhanced when a seal coating is applied to the core particles prior to drug layering.

The foregoing objects and advantages of the disclosure are illustrative of those that can be achieved by the various exemplary embodiments and are not intended to be exhaustive or limiting of the possible advantages which can be realized. Thus, these and other objects and advantages of the various exemplary embodiments will be apparent from the description herein or can be learned from practicing the various exemplary embodiments, both as embodied herein or as modified in view of any variation that may be apparent to those skilled in the art. Accordingly, the present disclosure relates to the novel methods, arrangements, combinations, and improvements herein shown and described in various exemplary embodiments.

### SUMMARY

In light of the present need for modified release skeletal muscle relaxant preparations which are less susceptible to active ingredient degradation resulting from contact with other ingredients, e.g., plasticizers, during storage and/or undesired release of active ingredient where modified release preparations are co-ingested with ethanol, a brief summary of various exemplary embodiments is presented. Some simplifications and omissions may be made in the following summary, which is intended to highlight and introduce some aspects of the various exemplary embodiments, but not to limit the scope of the invention. Detailed descriptions of preferred exemplary embodiments adequate to allow those of ordinary skill in the art to make and use the inventive concepts will follow in later sections.

In one aspect, the present disclosure relates to a multi-particulate pharmaceutical dosage form of a skeletal muscle relaxant providing a modified release profile comprising extended release beads, the extended release beads comprising: i) active-containing core particles comprising a skeletal muscle relaxant selected from the group consisting of cyclobenzaprine, dantrolene sodium, methocarbamol, metaxalone, carisoprodol, diazepam, their pharmaceutically acceptable salts or derivatives thereof, and mixtures thereof; and ii) an extended release coating substantially free of plasticizer, surrounding the core particles, comprising a) a water insoluble polymer selected from the group consisting of ethers of cellulose, esters of cellulose, ethyl cellulose, polyvinyl acetate, copolymers of acrylic and methacrylic acid esters with quaternary ammonium groups, pH-insensitive ammonio methacrylic acid copolymers, and mixtures thereof, and b) an optional water soluble, pharmaceutically acceptable channeling agent; wherein the dosage form when dissolution tested using United States Pharmacopoeia Apparatus 1 (Baskets @ 100 rpm) in 900 mL of water at 37° C. exhibits a drug release profile substantially corresponding to the following pattern: after 2 hours, no more than about 40% of the total active is released; after 4 hours, from about 40-65% of the total active is released; and after 8 hours, from about 60-85% of the total active is released, wherein the dosage form provides therapeutically effective plasma concentration over a period of 24 hours to treat muscle spasm associated with painful musculoskeletal conditions when administered to a patient in need thereof.

In another aspect, the present disclosure relates to a multi-particulate pharmaceutical dosage form of a skeletal muscle relaxant providing a modified release profile comprising extended release beads, the extended release beads comprising: i) active-containing core particles comprising a skeletal muscle relaxant selected from the group consisting of cyclobenzaprine, dantrolene sodium, methocarbamol, metaxalone, carisoprodol, diazepam, their pharmaceutically acceptable salts or derivatives thereof, and mixtures thereof; and ii) an ethanol-resistant extended release coating surrounding the core particles, comprising a) a water insoluble polymer and b) an optional water soluble, pharmaceutically acceptable channeling agent, wherein the dosage form when dissolution tested using United States Pharmacopoeia Apparatus 2 (Paddles @ 50 rpm) in 900 mL of 0.1N HCl containing 40% ethanol at 37° C. exhibits a drug release profile substantially corresponding to the following pattern: at 1 hour, no more than about 15%, for example, no more than about 10%, e.g. no more than about 5%, of the total active is released; and at 2 hours, no more than about 55%, for example, no more than about 40%, e.g. no more than about 30%, of the total active is released, wherein the dosage form provides therapeutically effective plasma concentration over a period of 24 hours to treat muscle spasm associated with painful musculoskeletal conditions when administered to a patient in need thereof.

In still another aspect, the present disclosure relates to a multi-particulate pharmaceutical dosage form of a skeletal muscle relaxant providing a modified release profile comprising extended release beads, the extended release beads comprising: i) active-containing core particles comprising a skeletal muscle relaxant selected from the group consisting of cyclobenzaprine, dantrolene sodium, methocarbamol, metaxalone, carisoprodol, diazepam, their pharmaceutically acceptable salts or derivatives thereof, and mixtures thereof; and ii) an extended release coating substantially free of plasticizer surrounding the core particles, comprising a) a water insoluble polymer selected from the group consisting of ethers of cellulose, esters of cellulose, ethyl cellulose, polyvinyl acetate, copolymers of acrylic and methacrylic acid esters with quaternary ammonium groups, pH-insensitive ammonio methacrylic acid copolymers, and mixtures thereof; and b) an optional water soluble, pharmaceutically acceptable channeling agent, wherein the dosage form when dissolution tested using United States Pharmacopoeia Apparatus 1 (Baskets @ 100 rpm) in 900 mL of water at 37° C. exhibits a drug release profile substantially corresponding to the following pattern: after 2 hours, no more than about 40% of the total active is released; after 4 hours, from about 40-65% of the total active is released; and after 8 hours, from about 60-85% of the total active is released, wherein the dosage form provides therapeutically effective plasma concentration over a period of 24 hours to treat muscle spasm associated with painful musculoskeletal conditions when administered to a patient in need thereof; and the dosage form exhibits at least about 90% dissolution stability in capsule form in percentage dissolved in water, after a three month storage period under 40° C. and 75% relative humidity, relative to a baseline of 100% dissolution in water as measured at the onset of the storage period.

In yet another aspect, the present disclosure relates to a multi-particulate pharmaceutical dosage form of a skeletal muscle relaxant providing a modified release profile comprising extended release beads, the extended release beads comprising: i) active-containing core particles comprising a skeletal muscle relaxant, cyclobenzaprine, pharmaceutically acceptable salts or derivatives thereof, and mixtures thereof; and ii) an extended release coating, surrounding the core particles, comprising a) a water insoluble polymer selected from the group consisting of ethers of cellulose, esters of cellulose, ethyl cellulose, polyvinyl acetate, copolymers of acrylic and methacrylic acid esters with quaternary ammonium groups, pH-insensitive ammonio methacrylic acid copolymers, and mixtures thereof; and b) an optional water soluble, pharmaceutically acceptable channeling agent, wherein the dosage form when dissolution tested using United States Pharmacopoeia Apparatus 1 (Baskets @ 100 rpm) in 900 mL of water at 37° C. exhibits a drug release profile substantially corresponding to the following pattern: after 2 hours, no more than about 40% of the total active is released; after 4 hours, from about 40-65% of the total active is released; and after 8 hours, from about 60-85% of the total active is released, wherein the dosage form provides therapeutically effective plasma concentration over a period of 24 hours to treat muscle spasm associated with painful musculoskeletal conditions when administered to a patient in need thereof.

In yet still another aspect, the present disclosure relates to a method of preparing a multi-particulate pharmaceutical dosage form of a skeletal muscle relaxant containing extended release beads providing a modified release profile, the method comprising: a) preparing unsealed active-containing core particles by applying to pharmaceutically inert particles a skeletal muscle relaxant selected from the group consisting of cyclobenzaprine, dantrolene sodium, methocarbamol, metaxalone, carisoprodol, diazepam, their pharmaceutically acceptable salts or derivatives thereof, and mixtures thereof; and b) preparing an extended release coating substantially free of plasticizer in a solution comprising i) a water insoluble polymer selected from the group consisting of ethers of cellulose, esters of cellulose, ethyl cellulose, polyvinyl acetate, pH-insensitive ammonio methacrylic acid copolymers, and mixtures thereof selected from the group consisting of ethers of cellulose, esters of cellulose, ethyl cellulose, polyvinyl acetate, copolymers of acrylic and methacrylic acid esters with quaternary ammonium groups, pH-insensitive ammonio methacrylic acid copolymers, and mixtures thereof; and ii) an optional water soluble, pharmaceutically acceptable channeling agent, and a substantially non-aqueous solvent for coating the active-containing core particles; and c) applying the extended release coating to the active-containing core particles to provide the extended release beads.

### BRIEF DESCRIPTION OF THE DRAWINGS

In order to better understand various exemplary embodiments, reference is made to the accompanying drawings, wherein:

FIG. 1 shows the target release profile for 30 mg cyclobenzaprine hydrochloride MR (modified release) capsules in water over 20 hours using USP Apparatus I (Baskets) at 100 rpm for embodiments of the present plasticizer-free invention having different levels of channeling agent (Examples 1-3) and a corresponding 30 mg capsule containing an extended-relase formulation of cyclobenzaprine having plasticizer in the extended release coating, currently marketed as Amrix® (available from Cephalon) having plasticizer in the extended release coating.

FIG. 2 shows the target release profile for 30 mg cyclobenzaprine hydrochloride MR (modified release) capsules in water over 20 hours using USP Apparatus I (basket) at 100 rpm for embodiments of the present plasticizer-free invention having three different viscocity grades of extended release polymer-ethylcellulose (45 cP), ethylcellulose (10 cP), and ethylcellulose (7 cP) (Examples 4-6) and a corresponding 30 mg capsule containing Amrix® (available from Cephalon) having plasticizer in the extended release coating.

FIG. 3 shows the target release profile for 30 mg cyclobenzaprine hydrochloride MR (modified release) capsules in water over 20 hours using USP Apparatus I (Baskets) at 100 rpm for embodiments of the present plasticizer-free invention having three different coating weight gains for the extended release coat, providing coated totals of 105.3% w/w, 110.3% w/w, and 115.3% w/w (with 100.0% core total) agent (Examples 7-9) and a corresponding 30 mg capsule containing Amrix® (available from Cephalon) having plasticizer in the extended release coating.

FIG. 4 shows the target release profile for 30 mg cyclobenzaprine hydrochloride MR (modified release) capsules in water over 20 hours using USP Apparatus I (Baskets) at 100 rpm for embodiments of the present plasticizer-free invention having two different channeling agents, hydroxypropyl cellulose and polyethylene oxide (Examples 10 and 11) and a corresponding 30 mg capsule containing Amrix® (available from Cephalon) having plasticizer in the extended release coating.

FIG. 5 shows the target release profile for 30 mg cyclobenzaprine hydrochloride MR (modified release) capsules in water over 20 hours using USP Apparatus I (Baskets) at 100 rpm for embodiments of the present plasticizer-free invention having two different channeling agents, hydroxypropyl cellulose and polyethylene oxide (Examples 10 and 11) and a corresponding 30 mg capsule containing Amrix® (available from Cephalon) having plasticizer in the extended release coating.

FIG. 6 depicts the target release profile showing dissolution stability for 30 mg cyclobenzaprine hydrochloride MR (modified release) capsules in water over 24 hours using USP Apparatus I (Baskets) for the product of EXAMPLE 1 after varied storage periods at 40° C./75% relative humidity for 0 month, one month, two month and three month periods, showing excellent dissolution stability even after three months of storage.

FIG. 7 shows the target release profile for 30 mg cyclobenzaprine hydrochloride MR (modified release) capsules in 0.1N HCl containing 40% alcohol over 120 minutes using USP Apparatus II (Paddle) at 50 rpm for the embodiments of the present plasticizer-free invention of Example 13 compared with a corresponding 30 mg capsule containing Amrix® (available from Cephalon) having plasticizer in the extended release coating. The results show improved resistance in vitro by a product of the present invention to alcohol induced dose dumping than the reference Amrix® product.

### DETAILED DESCRIPTION OF THE PREFERRED EMBODIMENTS

All documents cited are, in relevant part, incorporated herein by reference; the citation of any document is not to be construed as an admission that it is prior art with respect to the present disclosure.

The active core of the dosage form of the present disclosure may be comprised of an inert particle or an acidic or alkaline buffer crystal, which is coated with a drug-containing film-forming formulation and preferably a water-soluble film forming composition to form a water-soluble/dispersible particle. Alternatively, the active may be prepared by granulating and milling and/or by extrusion and spheronization of a polymer composition containing the drug substance. The amount of drug in the core will depend on the dose that is required, and typically varies from about 5 to about 60 weight %. Generally, the polymeric coating on the active core will be from about 4 to about 20% based on the weight of the coated particle, depending on the type of release profile required and/or the polymers and coating solvents chosen. Those skilled in the art will be able to select an appropriate amount of drug for coating onto or incorporating into the core to achieve the desired dosage. In one embodiment, the inactive core may be a sugar sphere or a buffer crystal or an encapsulated buffer crystal such as calcium carbonate, sodium bicarbonate, fumaric acid, tartaric acid, etc. which alters the microenvironment of the drug to facilitate its release.

The drug-containing particle may be coated with an extended release (ER) coating comprising a water insoluble polymer or a combination of a water insoluble polymer and a water soluble polymer to provide ER beads. In accordance with certain embodiments, the water insoluble polymer and the water soluble polymer may be present at a weight ratio of from about 100/0 to about 65/35, more particularly from about 95/5 to about 70/30, and still more particularly at a ratio of from about 85/15 to about 75/25. The extended release coating is applied in an amount necessary to provide the desired release profile. The extended release coating typically comprises from about 1% to about 15%, more particularly from about 5% to about 12%, by weight of the coated beads.

The present invention also provides a method of making a modified release dosage form. In accordance with one embodiment, the method includes the steps of: 1. preparing a drug-containing core by coating an inert particle such as a non-pareil seed, an acidic buffer crystal or an alkaline buffer crystal with a drug and a polymeric binder or by granulation and milling or by extrusion/spheronization to form an immediate release (IR) bead; 2. coating the IR bead with a coating formulation comprising plasticized water-insoluble polymer alone such as ethylcellulose or in combination with a water soluble polymer such as hydroxypropylmethylcellulose, in the absence of a plasticizer, to form an Extended Release (ER) bead; 3. filling into hard gelatin capsules the ER Beads to produce capsules providing the desired release profile.

Dissolution Procedure:

Dissolution Apparatus: USP Apparatus I (Baskets at 100 rpm), dissolution medium: 900 mL water (or a suitable dissolution medium) at 37° C. and Drug Release determination by HPLC.

An aqueous or a pharmaceutically acceptable solvent medium may be used for preparing drug-containing core particles. The type of film forming binder that is used to bind the drug to the inert sugar sphere is not critical but usually water soluble, alcohol soluble or acetone/water soluble binders are used. Binders such as polyvinylpyrrolidone (PVP), polyethylene oxide, hydroxypropyl methylcellulose (HPMC), hydroxypropylcellulose (HPC), polysaccharides such as dextrin, corn starch may be used at concentrations from about 0.5 to about 5 weight %, although other concentrations may be useful. The drug substance may be present in this coating formulation in the solution form or may be dispersed at a solid content up to about 35 weight % depending on the viscosity of the coating formulation.

In accordance with certain embodiments, the drug substance, optionally a binder such as PVP, a dissolution rate controlling polymer (if used), and optionally other pharmaceutically acceptable excipients are blended together in a planetary mixer or a high shear granulator such as Fielder and granulated by adding/spraying a granulating fluid such as water or alcohol. The wet mass can be extruded and spheronized to produce spherical particles (beads) using an extruder/marumerizer. In these embodiments, the drug load could be as high as 90% by weight based on the total weight of the extruded/spheronized core.

Representative muscle relaxants include cyclobenzaprine, dantrolene sodium, methocarbamol, metaxalone, carisoprodol, diazepam and pharmaceutically acceptable salts or derivatives thereof. Cyclobenzaprine hydrochloride is a particularly useful muscle relaxant. As used herein, the useful muscle relaxants include the base, pharmaceutically acceptable salts thereof such as hydrochloride, stereoisomers thereof and mixtures thereof.

The active ingredient in a commercialized CYCLOBENZAPRINE ER PRODUCT, e.g., AMRIX® (available from Cephalon) is cyclobenzaprine hydrochloride. Cyclobenzaprine hydrochloride is a white, crystalline tricyclic amine salt with the empirical formula C₂₀H₂₁N.HCl and a molecular weight of 311.9. It has a melting point of 217° C., and a pKa of 8.47 at 25° C. It is freely soluble in water and alcohol, sparingly soluble in isopropanol, and insoluble in hydrocarbon solvents. Cyclobenzaprine HCl is designated chemically as 3-(5H-dibenzo[a,d] cyclohepten-5-ylidene)-N,N-dimethyl-1-propanamine hydrochloride, and has the following structural formula:

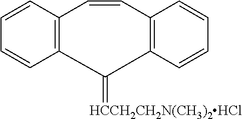

Representative examples of water insoluble polymers useful in the ER coating include ethylcellulose powder or an aqueous dispersion (such as AQUACOAT® ECD-30), polyvinyl acetate (Kollicoat SR#30D from BASF), copolymers of acrylic and methacrylic acid esters with quaternary ammonium groups such as Eudragit NE, RS and RS30D, RL or RL30D and the like. Representative examples of water soluble polymers useful herein include low molecular weight hydroxypropyl methylcellulose (HPMC), methylcellulose, hydroxypropylcellulose, polyvinylpyrrolidone (povidone), polyethylene glycol (PEG of molecular weight>3000) and mixtures thereof. The extended release coating will typically be applied at a thickness ranging from about 1 weight % up to about 15 weight % depending on the solubility of the active in water and the solvent or latex suspension based coating formulation used.

A plasticizer is a chemical additive that makes a plastic material softer and more pliable. Plasticizers used in polymer formulations that can be used in pharmaceutical dosage forms include triacetin, tributyl citrate, tri-ethyl citrate, acetyl tri-n-butyl citrate, diethyl phthalate, dibutyl sebacate, polyethylene glycol, polypropylene glycol, castor oil, acetylated mono- and di-glycerides and mixtures thereof.

The coating compositions used in forming the membranes of the present disclosure are typically not plasticized, in order to avoid undesired effects of plasticizer. Such effects can include reaction of plasticizer with active ingredient resulting in degradation of the active ingredient, and/or losses in storage stability, particularly under less than ideal conditions such as humid conditions, e.g., at least about 60%, e.g., at least about 75% relative humidity, and at greater than room temperatures, e.g., above about 32° C., above about 37° C., or even about 40° C. or greater. In certain embodiments, plasticizer may be present, but only in insignificant amounts which do not affect pliability of a coating, e.g., at less than about 1 wt. % and more typically at less than about 0.01 wt. % based on the polymer content of the coating.

In general, it is desirable to prime the surface of the particle before applying an extended release membrane coating or to separate the different membrane layers by applying a thin hydroxypropyl methylcellulose (HPMC)(OPADRY® Clear) film as a seal coating. While HPMC is typically used, other primers such as hydroxypropylcellulose (HPC) can also be used. In certain embodiments of the disclosure, the seal coating the core particles serves to enhance the potency of the active pharmaceutical ingredient, e.g., cyclobenzaprine HCl. In certain embodiments of the disclosure, seal coating the core particles is not necessary.

The membrane coatings can be applied to the core using any of the coating techniques commonly used in the pharmaceutical industry, but fluid bed coating is particularly useful.

The present invention is applied to multi-dose forms, i.e., drug products in the form of multi-particulate dosage forms (pellets, beads, granules or mini-tablets) or in other forms suitable for oral administration. As used herein, these terms are used interchangeably to refer to multi-particulate dosage forms.

The invention also provides a method of making an extended release dosage form. In accordance with one aspect of the present invention, the method includes the steps of: (a) coating an inert particle such as a non-pareil seed, an acidic buffer crystal or an alkaline buffer crystal with a drug and polymeric binder to form an active drug particle; (b) coating the active drug particle with a solution or suspension of a water insoluble polymer or a mixture of water soluble and water insoluble polymers to form an extended release coated drug particle (ER beads); and (c) filling into a hard gelatin capsule ER beads to produce a MR (modified release) capsule exhibiting a target drug release profile.

Unintended, rapid drug release in a short period of time of the entire amount or a significant fraction of the drug contained in a modified release dosage form is often referred to as “dose dumping.” Depending on the therapeutic indication and the therapeutic index of a drug, dose-dumping can pose a significant risk to patients, either due to safety issues or diminished efficacy or both. Generally dose-dumping is observed due to a compromise of the release-rate-controlling mechanism. The likelihood of dose-dumping for certain modified release products when administered with food has been recognized for about twenty years and a regulatory process has been established to address it. Similar concerns associated with coadministration of modified release products and alcohol are being addressed by regulators as well.

Some modified-release oral dosage forms contain drugs and excipients that exhibit higher solubility in ethanolic solutions compared to water. Such products can be expected to exhibit a more rapid drug dissolution and release rate in the presence of ethanol. Therefore, in theory, concomitant consumption of alcoholic beverages along with these products might be expected to have the potential to induce dose dumping. This potential mechanism leading to dose-dumping from an oral modified-release dosage form has not previously attracted attention in the pharmaceutical science literature or in regulatory assessment process. It is now appreciated that consumption of alcohol with administration of modified release formulations can cause excessive concentrations of active ingredient in a patient's blood stream. Both in vitro and in vivo testing measures have been developed to determine the susceptibility of modified release formulations to “dose dumping” by co-ingestion of modified-release formulation and ethanol.

In certain embodiments, the modified-release formulations of the present disclosure exhibit resistance to dose dumping. Such resistance is observable where a formulation includes an ethanol-resistant extended release coating surrounding the core particles, which comprises a water insoluble polymer in the substantial absence of plasticizer. Such resistance has been confirmed with clinically relevant effects documented in vivo as shown in the Examples below.

In certain embodiments of the disclosure, the channeling agent comprises a water soluble single-molecule compound selected from the group consisting of single-molecule inorganic salts, sugars, sugar alcohols, water soluble organic salts and mixtures thereof, alone or in combination with water soluble polymer. Such water soluble polymer is selected from the group consisting of hydroxypropyl cellulose, hydroxypropyl methylcellulose, hydroxyethyl cellulose, povidone, polyethylene oxide, polyethylene glycol, and mixtures thereof. In some embodiments of the disclosure, the channeling agent is selected from the group consisting of sodium chloride, sodium phosphate, sucrose, lactose, dextrose, mannitol, xylitol, sorbitol, maltitol, sodium acetate, sodium citrate, hydroxypropyl cellulose, hydroxypropyl methylcellulose, hydroxyethyl cellulose, povidone, polyethylene oxide, polyethylene glycol, and mixtures thereof.

In some embodiments of the disclosure, the skeletal muscle relaxant is cyclobenzaprine hydrochloride. In certain embodiments, the pharmaceutical dosage form provides a maximum blood plasma concentration (Cₘₐₓ) within the range of about 80% to 125% of about 20 ng/mL of cyclobenzaprine HCl and an AUC₀₋ₜ within the range of about 80% to 125% of about 740 nghr/mL following oral administration of a single 30 mg cyclobenzaprine HCl MR Capsule.

In certain embodiments of the disclosure, the water insoluble polymer on the core particles comprises from about 5% to about 20% by weight of the extended release beads, e.g., from about 5% to about 12% by weight of the extended release beads.

In some embodiments of the disclosure, the water soluble channeling agent comprises a water soluble polymer selected from the group consisting of methylcellulose, hydroxypropylcellulose, hydroxypropyl methylcellulose, polyethylene glycol, povidone, and mixtures thereof, e.g., the water insoluble polymer comprises ethyl cellulose. In some embodiments, the ethyl cellulose has a viscosity ranging from about 5 to about 50 centipoise. In specific embodiments, the ethyl cellulose has a viscosity ranging from about 20 to about 45, e.g., from about 40 to about 50 centipoise. In other specific embodiments, the ethyl cellulose has a viscosity ranging from about 6 to about 12 centipoise.

In some embodiments, the extended release coating comprises the water soluble channeling agent selected from the group consisting of hydroxypropyl cellulose and polyethylene oxide.

In certain embodiments, the pharmaceutical dosage form of the present disclosure contains no added plasticizer.

In other embodiments, the pharmaceutical dosage form contains no greater than about 1 wt. % of plasticizer selected from the group consisting of triacetin, tributyl citrate, tri-ethyl citrate, acetyl tri-n-butyl citrate, diethyl phthalate, dibutyl sebacate, polyethylene glycol, polypropylene glycol, castor oil, acetylated mono- and di-glycerides and mixtures thereof, e.g., no greater than about 0.1 wt. %, or even no greater than about 0.01 wt. % of plasticizer.

In some embodiments of the present disclosure, the drug release profile substantially corresponds to the following pattern: after 2 hours, no more than about 40% of the total active is released; after 4 hours, from about 40-65% of the total active is released; after 8 hours, from about 60-85% of the total active is released; and after 12 hours, from about 75-85% of the total active is released.

In certain embodiments of the present disclosure there is no water soluble, single-molecule, pharmaceutically acceptable channeling agent.

In some embodiments of the present disclosure, the extended release coating is substantially free of triacetin, tributyl citrate, tri-ethyl citrate, acetyl tri-n-butyl citrate, diethyl phthalate, dibutyl sebacate, polyethylene glycol, polypropylene glycol, castor oil, and acetylated mono- and di-glycerides, and mixtures thereof.

In certain embodiments of the present disclosure, the core particles are seal coated, e.g., with a coating formulation containing hydroxypropyl methylcellulose.

In some embodiments of the present disclosure, the pharmaceutical dosage form exhibits a Cₘₐₓ below 8.0 ng/mL in vivo when co-administered to an adult as a capsule in a dosage form of 15 mg with eight ounces of 20 vol. % ethanol.

In other embodiments, the pharmaceutical dosage form exhibits a Cₘₐₓ below 10.0 ng/mL in vivo when co-administered to an adult as a capsule in a dosage form of 15 mg with eight ounces of 40 vol. % ethanol.

In certain embodiments of the present disclosure, the water insoluble polymer is present within a membrane, typically, a membrane surrounding the core particle. The membrane can include additional components, e.g., additional components selected from talc and hydroxylpropyl methyl cellulose.

In other embodiments of the present disclosure, the method of preparing a multi-particulate pharmaceutical dosage form of a skeletal muscle relaxant containing extended release beads providing a modified release profile is carried out without exposure to curing conditions to provide the extended release beads. Such undesirable curing conditions include conditions which can cause degradation of the active ingredient, for example, temperatures greater than about 23° C., e.g., greater than about 37° C., or even greater than about 60° C., and exposure periods greater than about 0.5 hour, e.g., greater than about 2 hours.

In some embodiments, the substantially non-aqueous solvent used in the method of preparation is selected from a volatile organic solvent selected from the group consisting of ethanol, isopropanol, acetone and methylene chloride, e.g., the substantially non-aqueous solvent comprises ethanol.

In some embodiments of the present disclosure, the method of preparing is carried out wherein step c) comprises directly applying the extended release coating to the active-containing core particles to provide the extended release beads.

In certain embodiments of the present disclosure, the method of preparing further comprises: d) encapsulating the extended release beads in gel capsules to provide the dosage forms.

In other embodiments of the present disclosure, the method of preparing is carried out wherein the core particles are seal coated prior to step c), e.g., with a coating formulation containing hydroxypropyl methylcellulose.

In selected embodiments of the present disclosure, the method of preparing is carried out wherein the core particles are not seal coated prior to step c).

The following non-limiting examples illustrate the capsule dosage forms manufactured in accordance with the disclosure using cyclobenzaprine hydrochloride as a test case, which exhibit in vitro or in vivo drug release profiles. Such dosage forms when orally administered, would enable maintaining drug plasma concentrations at therapeutically effective levels over extended periods of time, thereby significantly improving patient compliance.

### EXAMPLES 1-3

### Examples of the Disclosure with Different Levels of Water Soluble Channeling Agent

### EXAMPLE 1

In a fluid bed coater with Wurster insert, 32 kg of sugar spheres 20/25 were coated with 12.8 kg of Clear Opadry® solution in water (10% solid content) followed by coating with 90 kg of drug layering suspension in 80/20 isopropanol (IPA)/Water (15% solid content) containing cyclobenzaprine HCl, hypromellose 2910 (3 centipoise (cPs)) and talc. The drug-containing core beads were then coated with 100.3 g of extended-release coating solution in alcohol (7% solid content) containing ethylcellulose (45 cPs) and hypromellose 2910 (6 cPs). After the extended-release coated beads were blended with 160 g of talc and screened, the acceptable ER beads were encapsulated into capsules containing 15 mg or 30 mg cyclobenzaprine HCl.

### EXAMPLES 2 AND 3

EXAMPLES 2 and 3 were prepared using the same procedure as EXAMPLE 1.

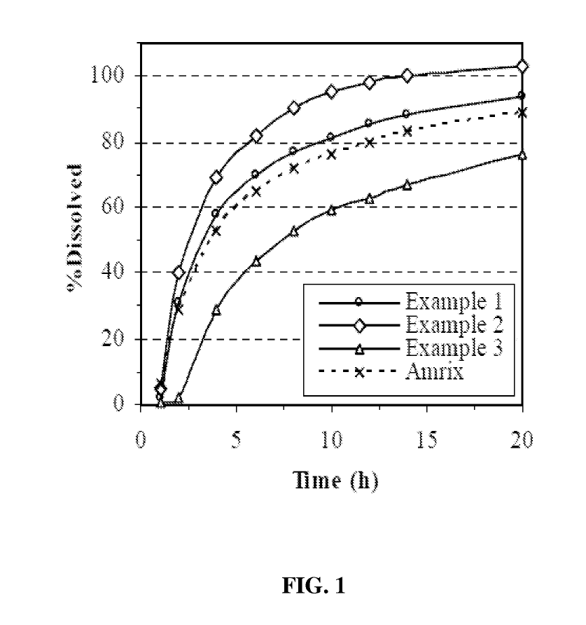

Dissolution of Cyclobenzaprine HCl, 30 mg in water using USP Apparatus I (Baskets) at 100 RPM was carried out for EXAMPLES 1-3 and compared with that of a commercially available 30 mg Amrix® capsule which contains plasticizer. The dissolution profile is set out in FIG. 1 below.

The constituents of Examples 1-3 are set out below in TABLE 1.

TABLE 1
Examples of Cyclobenzaprine ER Formulations - Different
Levels of Channeling Agent

| | % w/w | | |
|---|---|---|---|
| Ingredients | Example 1 | Example 2 | Example 3 |
| Active-containing core: | | | |
| Cyclobenzaprine HCl | 17.3 | 17.3 | 17.3 |
| Sugar Sphere 20/25 | 68.4 | 68.4 | 68.4 |
| Clear Opadry (YS-1-7006) | 2.74 | 2.74 | 2.74 |
| Hypromellose 2910 (3 cPs) | 5.77 | 5.77 | 5.77 |
| Talc | 5.77 | 5.77 | 5.77 |
| Core Total: | 100.0 | 100.0 | 100.0 |
| ER coat: | | | |
| Ethylcellulose (45 cPs) | 9.0 | 8.55 | 12.0 |
| Hypromellose (6 cPs) | 6.0 | 6.45 | 3.0 |
| Talc | 0.3 | 0.3 | 0.3 |
| Coated Total: | 115.3 | 115.3 | 115.3 |

### EXAMPLES 4-6

### Examples of the Invention with Different Viscosity Grades of Extended-Release Polymer

EXAMPLES 4-6 were prepared according to the same procedure set out in EXAMPLE 1, but with the ingredients set out in TABLE 2 below.

TABLE 2
Examples of Cyclobenzaprine ER Formulations - Different Viscosity
Grades of Extended-release Polymer

| | % w/w | | |
|---|---|---|---|
| Ingredients | Example 4 | Example 5 | Example 6 |
| Active-containing core: | | | |
| Cyclobenzaprine HCl | 17.3 | 17.3 | 17.3 |
| Sugar Sphere 20/25 | 68.4 | 68.4 | 68.4 |
| Clear Opadry (YS-1-7006) | 2.74 | 2.74 | 2.74 |
| Hypromellose 2910 (3 cPs) | 5.77 | 5.77 | 5.77 |
| Talc | 5.77 | 5.77 | 5.77 |
| Core Total: | 100.0 | 100.0 | 100.0 |
| ER coat: | | | |
| Ethylcellulose (45 cPs) | 6.375 | — | — |
| Ethylcellulose (10 cPs) | — | 6.375 | — |
| Ethylcellulose (7 cPs) | — | — | 6.375 |
| Hypromellose (6 cPs) | 1.125 | 1.125 | 1.125 |
| Talc | 0.3 | 0.3 | 0.3 |
| Coated Total: | 107.8 | 107.8 | 115.3 |

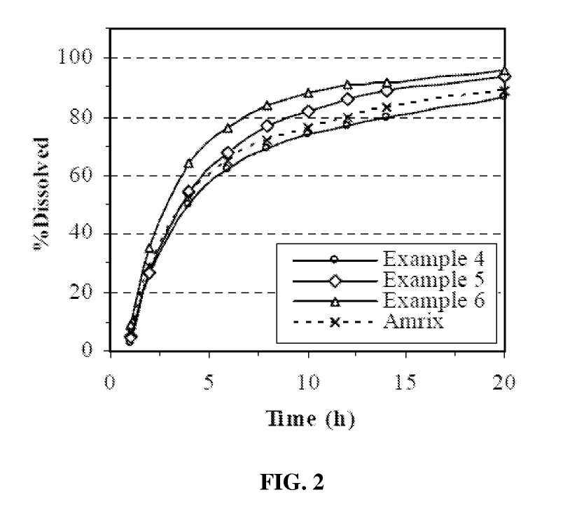

Dissolution of Cyclobenzaprine HCl, 30 mg in water using USP Apparatus I (Basket) at 100 RPM was carried out for EXAMPLES 4-6 which differ by having different viscosity grades of ethylcellulose water insoluble polymer and compared with that of a commercially available 30 mg Amrix® capsule. The dissolution profile is set out in FIG. 2 below.

### EXAMPLES 7-9

### Examples of the Invention with Different Coating Weight Gains

EXAMPLES 7-9 were prepared according to the same procedure set out in EXAMPLE 1, but with the ingredients set out in TABLE 3 below.

TABLE 3
Examples of Cyclobenzaprine ER Formulations - Different
Coating Weight Gains

| | % w/w | | |
|---|---|---|---|
| Ingredients | Example 7 | Example 8 | Example 9 |
| Active-containing core: | | | |
| Cyclobenzaprine HCl | 17.3 | 17.3 | 17.3 |
| Sugar Sphere 20/25 | 68.4 | 68.4 | 68.4 |
| Clear Opadry (YS-1-7006) | 2.74 | 2.74 | 2.74 |
| Hypromellose 2910 (3 cPs) | 5.77 | 5.77 | 5.77 |
| Talc | 5.77 | 5.77 | 5.77 |
| Core Total: | 100.0 | 100.0 | 100.0 |
| ER coat: | | | |
| Ethylcellulose (10 cPs) | 4.25 | 8.5 | 12.75 |
| Hypromellose (6 cPs) | 0.75 | 1.5 | 2.25 |
| Talc | 0.3 | 0.3 | 0.3 |
| Coated Total: | 105.3 | 110.3 | 115.3 |

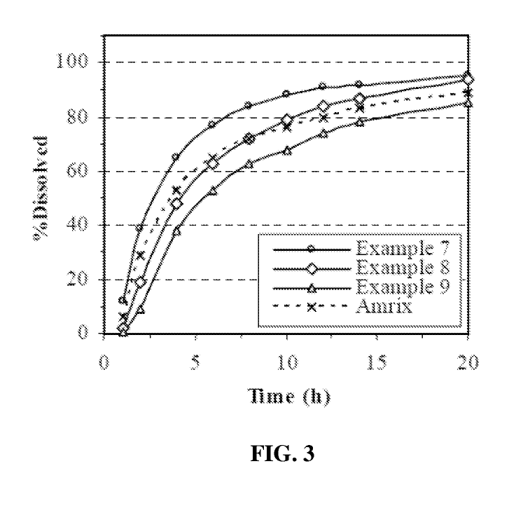

Dissolution of Cyclobenzaprine HCl, 30 mg in water using USP Apparatus I (Baskets) at 100 RPM was carried out for EXAMPLES 7-9 which differ by having different coating weight gains and compared with that of a commercially available 30 mg Amrix® capsule. The dissolution profile is set out in FIG. 3 below.

### EXAMPLES 10-11

### Examples of the Invention with Different Water Soluble Channeling Agents

EXAMPLES 10 AND 11 were prepared according to the same procedure set out in EXAMPLE 1, but with the ingredients set out in TABLE 4 below.

TABLE 4
Examples of Cyclobenzaprine ER Formulations - Different
Channeling Agents

% w/w

| Ingredients | Example 10 | Example 11 |
|---|---|---|
| Active-containing core: | | |
| Cyclobenzaprine HCl | 17.3 | 17.3 |
| Sugar Sphere 20/25 | 68.4 | 68.4 |
| Clear Opadry (YS-1-7006) | 2.74 | 2.74 |
| Hypromellose 2910 (3 cPs) | 5.77 | 5.77 |
| Talc | 5.77 | 5.77 |
| Core Total: | 100.0 | 100.0 |
| ER coat: | | |
| Ethylcellulose (10 cPs) | 10.625 | 10.625 |
| Hydroxypropyl Cellulose (EF) | 1.875 | — |
| Polyethylene Oxide (N10) | — | 1.875 |
| Talc | 0.3 | 0.3 |
| Coated Total: | 112.8 | 112.8 |

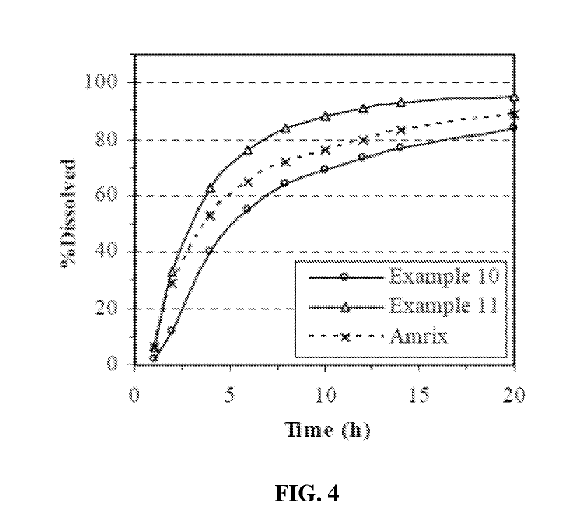

Dissolution of Cyclobenzaprine HCl, 30 mg in water using USP Apparatus I (Basket) at 100 RPM was carried out for EXAMPLES 10 and 11 which differ by having different water soluble channeling agents (hydroxypropyl cellulose versus polyethylene oxide) and compared with that of a commercially available 30 mg Amrix® capsule. The dissolution profile is set out in FIG. 4 below.

### EXAMPLES 12-13

### Examples of the Invention Lacking Water Soluble Channeling Agents and Containing Varying Amounts of Water Insoluble Polymer (Ethylcellulose (10 Cp))

EXAMPLES 12 AND 13 were prepared according to the same procedure set out in EXAMPLE 1, but with the ingredients set out in TABLE 5 below.

TABLE 5
Examples of Cyclobenzaprine ER Formulations - Without
Channeling Agent

% w/w

| Ingredients | Example 12 | Example 13 |
|---|---|---|
| Active-containing core: | | |
| Cyclobenzaprine HCl | 17.3 | 17.3 |
| Sugar Sphere 20/25 | 68.4 | 68.4 |
| Clear Opadry (YS-1-7006) | 2.74 | 2.74 |
| Hypromellose 2910 (3 cPs) | 5.77 | 5.77 |
| Talc | 5.77 | 5.77 |
| Core Total: | 100.0 | 100.0 |
| ER coat: | | |
| Ethylcellulose (10 cPs) | 7.0 | 10.0 |
| Talc | 0.3 | 0.3 |
| Coated Total: | 107.3 | 110.3 |

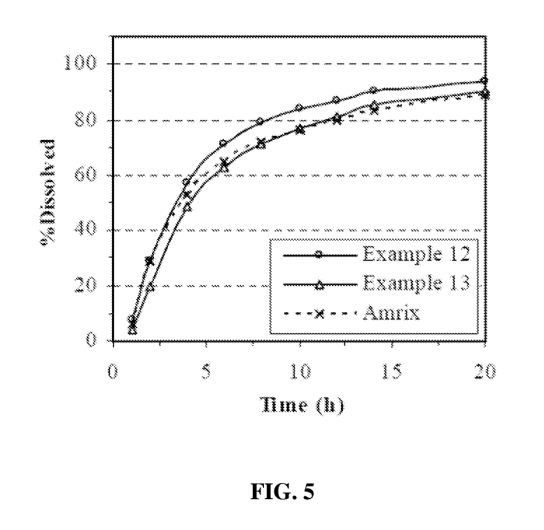

Dissolution of Cyclobenzaprine HCl, 30 mg in water using USP Apparatus I (Basket) at 100 RPM was carried out for EXAMPLES 4-6 which lack water soluble channeling agents and which contain varied amounts of insoluble polymer (ethylcellulose (10 cP)) in the coating. The two examples were also compared with that of a commercially available 30 mg Amrix® capsule. The dissolution profile is set out in FIG. 5 below.

### EXAMPLE 14

### Comparison of Er Coating Strengths During Dissolution for the Present Invention Formulations and Commercially Available Amrix® (Containing Plasticizer)

The ER coating system without plasticizer of the present invention surprisingly withstands attritions from the fluid bed coating process and retains membrane integrity during dissolution over 24 hours. This is indicated by the low variation (RSD) of the dissolution data from 12 dissolution vessels using samples from EXAMPLE 1 as compared to those of the commercially available reference product AMRIX® (obtained from Cephalon) which contains plasticizer, as set out below in TABLE 6.

TABLE 6
Dissolution Results^{a}

| | Time | | |
|---|---|---|---|
| | 2 hr | 4 hr | 8 hr |
| Example 1 - | 31% (2.6%) | 56% (2.3%) | 77% (2.4%) |
| Coating without | | | |
| plasticizer | | | |
| Amrix - | 29% (5.6%) | 53% (3.8%) | 72% (2.9%) |
| Coating with plasticizer | | | |

^{a}Conditions used: Apparatus 1 (Baskets) @ 100 rpm in 900 mL water at 37° C. ± 5° C.;
Values given are the mean of 12 duplicate runs and RSD data are in parenthesis.

### Example 15

### Comparison of Respective Dissolution Stability During Storage for a Present Invention Formulation and a Commercially Available Amrix® Containing Plasticizer

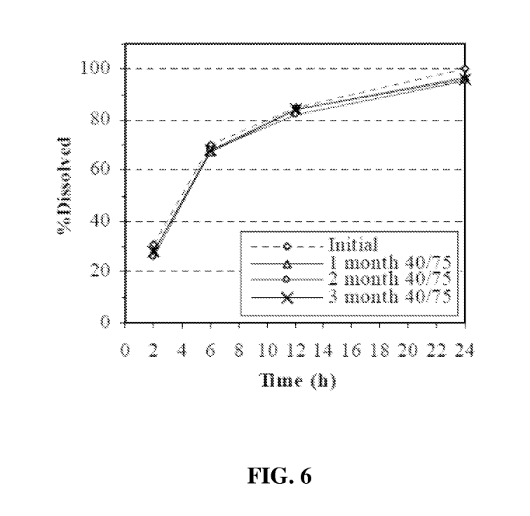

The ER coating system without plasticizer of the present invention exhibits excellent dissolution stability over three months of storage, under conditions which include temperatures of 40° C. at 75% relative humidity, using a sample from EXAMPLE 1 above. The lack of plasticizer avoids the problem of plasticizer leaching out of the membrane coating during storage, even under rigorous storage conditions. Dissolution stabilities were measured prior to storage, and at one month intervals up to three months. The dissolution profiles are set out below in FIG. 6 and show little variability over three months of storage.

### Example 16

### Comparison of Respective Dissolution Profiles in Ethanol Solutions for the Present Invention Formulations and Commercially Available Amrix® Containing Plasticizer

Current FDA policy requires testing of modified-release products to identify the potential for “dose-dumping” in the presence of alcohol prior to approval. The ER coating system of the invention without plasticizer showed excellent resistance to alcohol induced dose dumping as demonstrated by in vivo alcohol dose dumpling studies described below using the formulation of EXAMPLE 4 in comparison with the reference product AMRIX®.

The in vivo effect of alcohol (20 vol. % and 40 vol. %) co-administration on the disposition of Cyclobenzaprine Extended-release (CER) Capsules (15 mg) from Example 4 and corresponding AMRIX® Extended-release (CER) Capsules (15 mg) was studied.

After obtaining written informed consent, 22 healthy, adult alcohol tolerant subjects (18 males) weighing 93±18 kg and 28±7 years of age participated in this open-label, single-dose, randomized, four-period, four-treatment crossover study. For subject safety, the order of alcohol dosing was fixed with the 20 vol. % alcohol solution (in apple juice) given during Periods 1 & 2 and the 40 vol. % alcohol solution given during Periods 3 & 4. After an overnight fast, subjects received an oral dose of CER with 240 mL of alcohol over 20 min. Blood samples were taken pre-dose and until 96 hours post-dose. Plasma was assayed using a validated LC/MS/MS methodology. Analysis of the data used a standard FDA bioequivalence approach.

Twenty (16 males) subjects completed study Periods 1 & 2 and 16 (13 males) subjects completed study Periods 3 and 4. The results are presented in Table 7 below. In this and subsequent Tables, AUCL is the area under the concentration time curve from time zero to the last measurable concentration AUC_{0 to t}, which is interchangeable with AUCL. Based on these results it appears the extended-release CER capsules (15 mg) made in accordance with the invention and the AMRIX® extended-release (CER) Capsules (15 mg) are equivalent when co-administered with alcohol.

TABLE 7
The effect of alcohol co-administration on the disposition of
cyclobenzaprine in alcohol tolerant adult volunteers

| | | | | |
|---|---|---|---|---|
| | Mean (% CV) | Mean(% CV) | LSMEANS | 90% Confidence |
| Parameter | A = Example 4 | B = Amrix ® | Ratio (A/B) | Interval |

Cyclobenzaprine and 20% alcohol (n = 20)

| | | | | |
|---|---|---|---|---|
| AUC₀₋ₜ (ng × hr/mL) | 232.97 (36.16%) | 239.89 (37.57%) | 0.98 | 93.73%-102.84% |
| AUC_{inf} (ng × hr/mL) | 272.39 (38.67%) | 280.87 (38.46%) | 0.98 | 93.70%-101.46% |
| C_{MAX} (ng/mL) | 7.62 (31.17%) | 8.18 (30.06%) | 0.95 | 87.72%-102.60% |

Cyclobenzaprine and 40% alcohol (n = 16)

| | | | | |
|---|---|---|---|---|
| AUC₀₋ₜ (ng × hr/mL) | 293.51 (36.82%) | 309.24 (36.21%) | 0.96 | 87.25%-104.83% |
| AUC_{inf} | 347.70 (40.13%) | 355.55 (36.46%) | 0.98 | 89.65%-106.99% |
| (ng × hr/mL) | | | | |
| C_{MAX} (ng/mL) | 9.88 (38.33%) | 11.59 (40.85%) | 0.86 | 80.05%-92.62% |

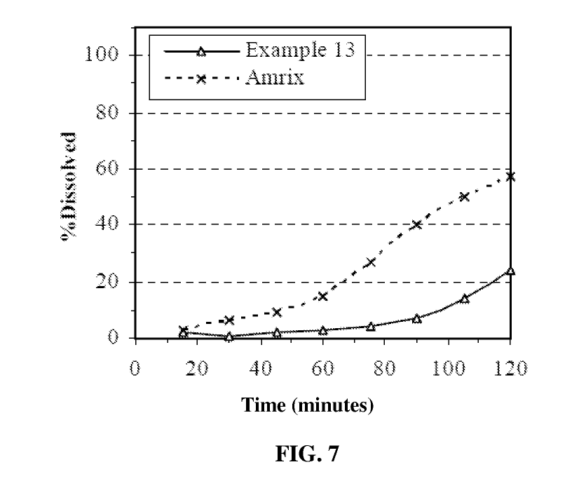

The dissolution profile set out in FIG. 7 below compares a sample of the invention from EXAMPLE 13 with the reference product. As can be seen in FIG. 7, in some instances, the products of the invention showed better resistance in vitro to alcohol induced dose dumping than the reference product AMRIX®. In particular, FIG. 7 shows the target release profile for 30 mg cyclobenzaprine hydrochloride MR (modified release) capsules in 0.1N HCl containing 40% alcohol over 120 minutes using USP Apparatus II (Paddle) at 50 rpm for the embodiments of the present plasticizer-free invention compared with a corresponding 30 mg capsule containing Amrix® (available from Cephalon) having plasticizer in the extended release coating. The results show improved resistance in vitro by a product of the present invention to alcohol induced dose dumping than the reference Amrix® product.

### Example 16

### Comparison of Respective Bioavailability for the Present Invention Formulations and Commercially Available Amrix® Containing Plasticizer

Cyclobenzaprine Extended-release Capsules, 30 mg using the ER coating system of the said invention without plasticizer demonstrated comparable biovailability to the reference product, AMRIX® with a plasticizer-based ER coating system, under both fasting and fed conditions.

Test conditions followed FDA guidelines as set out in the “Dissolution Methods Database” available to the public at the OGD website at http://www.fda.gov/cder/ogd/index.htm for conducting comparative dissolution testing on 12 dosage units each of all strengths of the test and reference products.

Testing Conditions were: 900 mL, 0.1N HCl, USP apparatus 2 (paddle) @ 50 rpm, with or without alcohol; Test 1: 12 units tested according to the proposed method (with 0.1N HCl), with data collected every 15 minutes for a total of 2 hours. Test 2: 12 units analyzed by substituting 5% (v/v) of test medium with Alcohol USP and data collection every 15 minutes for a total of 2 hours. Test 3: 12 units analyzed by substituting 20% (v/v) of test medium with Alcohol USP and data collection every 15 minutes for a total of 2 hours. Test 4: 12 units analyzed by substituting 40% (v/v) of test medium with Alcohol USP and data collection every 15 minutes for a total of 2 hours. The results of the bioequivalence studies are set out below in TABLE 8.

TABLE 8
Results of Bioequivalence Studies on Cyclobenzaprine Extended-release
Capsules (Example 1) and Amrix ®

| Active-Containing Core | % w/w | | |
|---|---|---|---|
| Ingredients | Example 14 | Example 1 | Example 13 |
| Seal Coating: | | | |
| Sugar Sphere 20/25 | 79.3 | 68.4 | 68.4 |
| Clear Opadry (YS-1-7006) | — | 2.74 | — |
| Hyprocellose 2910 (6 cP) | — | | 2.74 |
| Drug Layering: | | | |
| Cyclobenzaprine HCl | 18.0 | 17.3 | 17.3 |
| Hypromellose 2910 (3 cP) | 2.7 | 5.77 | 5.77 |
| Talc | — | 5.77 | 5.77 |
| Core Total: | 100.0 | 100.0 | 100.0 |
| Potency (% Theoretical Drug | 90.6 | 101.3 | 98.9 |
| Content) | | | |

### Example 17

### Effects of Seal Coating on Core Particle Potency for Present Invention Formulations

The effect of seal coating for cyclobenzaprine extended-release formulations of Examples 1, 13 and 14 of the present invention were tested and the results are set out in Table 9 below. It was found that the presence of a seal coating provided an unexpected increase in potency (% theoretical drug content) as measured by conventional techniques known to those of skill in the art.

TABLE 9
Examples of Cyclobenzaprine ER Formulations - Effect of
Seal Coating on Core Potency

| | | | | |
|---|---|---|---|---|
| | Mean | Mean | | |
| | A = | B = | LSMEANS | 90% Confidence |
| Parameter | Example 1 | Amrix ® | Ratio (A/B) | Interval |

Fasting (n = 23)

| | | | | |
|---|---|---|---|---|
| AUC₀₋ₜ | 500.57 | 474.45 | 1.05 | 99.77%-111.36% |
| (ng × hr/mL) | | | | |
| AUCinf | 542.56 | 515.52 | 1.06 | 99.86%-112.07% |
| (ng × hr/mL) | | | | |
| CPEAK | 16.28 | 14.45 | 1.12 | 107.33%- [symbol] |
| (ng/mL) | | | | |

Fed (n = 24)

| | | | | |
|---|---|---|---|---|
| AUC₀₋ₜ | 592.04 | 580.70 | 1.02 | 96.17%-108.09% |
| (ng × hr/mL) | | | | |
| AUCinf | 639.54 | 626.07 | 1.02 | 95.94%-108.76% |
| (ng × hr/mL) | | | | |
| CPEAK | 17.59 | 17.55 | 1.00 | 92.38%-108.40% |
| (ng/mL) | | | | |

[symbol]

indicates data missing or illegible when filed

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A multi-particulate pharmaceutical dosage form of a skeletal muscle relaxant providing a modified release profile comprising extended release beads, the extended release beads comprising:
i) active-containing core particles comprising a skeletal muscle relaxant selected from the group consisting of cyclobenzaprine, dantrolene sodium, methocarbamol, metaxalone, carisoprodol, diazepam, their pharmaceutically acceptable salts or derivatives thereof, and mixtures thereof; and
ii) an extended release coating substantially free of plasticizer, surrounding the core particles, said extended release coating consisting of:
a) a water insoluble polymer selected from the group consisting of ethers of cellulose, esters of cellulose, ethyl cellulose, polyvinyl acetate, copolymers of acrylic and methacrylic acid esters with quaternary ammonium groups, pH-insensitive ammonia methacrylic acid copolymers, and mixtures thereof,
b) an optional water soluble, pharmaceutically acceptable channeling agent, and
c) an optional filler:
wherein the dosage form when dissolution tested using United States Pharmacopoeia Apparatus 1 (Baskets @ 100 rpm) in 900 mL of water at 37° C. exhibits a drug release profile characterized in that after 2 hours, no more than about 40% of the total active is released;
wherein the dosage form when dissolution tested using United States Pharmacopoeia Apparatus 2 (paddles @50 rpm) in 900 mL of 0.1N HCl containing 40% ethanol at 37° C. exhibits a drug release profile characterized in that after 2 hours no more than about 55% of the total active is released; and
wherein the dosage form provides therapeutically effective plasma concentration over a period of 24 hours to treat muscle spasm associated with painful musculoskeletal conditions when administered to a patient in need thereof.

2. The pharmaceutical dosage form of claim 1, wherein the skeletal muscle relaxant is cyclobenzaprine hydrochloride.

3. The pharmaceutical dosage form of claim 2 wherein the pharmaceutical dosage form is a single capsule containing 30 mg cyclobenzaprine hydrochloride; and
wherein said pharmaceutical dosage form provides a maximum blood plasma concentration (Cₘₐₓ) within the range of about 80% to about 125% of about 20 ng/mL of cyclobenzaprine HCl and an AUC₀₋ₜ within the range of about 80% to about 125% of about 740 nghr/mL following oral administration of said capsule.

4. The pharmaceutical dosage form of claim 8, which contains no added plasticizer.

5. The pharmaceutical dosage form of claim 8, which contains no greater than about 1 wt. % of plasticizer selected from the group consisting of triacetin, tributyl citrate, tri-ethyl citrate, acetyl tri-n-butyl citrate, diethyl phthalate, dibutyl sebacate, polyethylene glycol, polypropylene glycol, castor oil, acetylated mono- and di-glycerides and mixtures thereof.

6. The pharmaceutical dosage form of claim 5, which contains no greater than about 0.1 wt. % of plasticizer.

7. The pharmaceutical dosage form of claim 1, wherein the core particles are seal coated with a coating formulation containing hydroxypropyl methylcellulose.

8. A multi-particulate pharmaceutical dosage form of a skeletal muscle relaxant providing a modified release profile comprising extended release beads, the extended release beads comprising:
i) active-containing core particles comprising a skeletal muscle relaxant selected from the group consisting of cyclobenzaprine, dantrolene sodium, methocarbamol, metaxalone, carisoprodol, diazepam, their pharmaceutically acceptable salts or derivatives thereof, and mixtures thereof; and
ii) an ethanol-resistant extended release coating surrounding the core particles, comprising a) a water insoluble polymer and b) an optional water soluble, pharmaceutically acceptable channeling agent, wherein the dosage form when dissolution tested using United States Pharmacopoeia Apparatus 2 (paddles @ 50 rpm) in 900 mL of 0.1N HCl containing 40% ethanol at 37° C. exhibits a drug release profile in an aqueous solution comprising at least about 40% ethanol substantially corresponding to the following pattern: at 1 hour, no more than about 15% of the total active is released; and at 2 hours, no more than about 55% of the total active is released, wherein the dosage form provides therapeutically effective plasma concentration over a period of 24 hours to treat muscle spasm associated with painful musculoskeletal conditions when administered to a patient in need thereof.

9. The dosage form of claim 8 wherein at 1 hour, no more than about 10% of the total active is released; at 1.5 hours, no more than about 30% of the total active is released;
and at 2 hours, no more than about 40% of the total active is released.

10. The dosage form of claim 9 wherein at 1 hour no more than about 5% of the total active is released; at 1.5 hours no more than about 15% of the total active is released; and at 2 hours, no more than about 30% of the total active is released.

11. The pharmaceutical dosage form of claim 8, wherein the skeletal muscle relaxant is cyclobenzaprine hydrochloride, the water insoluble polymer is selected from the group consisting of ethyl cellulose, polyvinyl acetate, copolymers of acrylic and methacrylic acid esters with quaternary ammonium groups, pH-insensitive ammonio methacrylic acid copolymers, and mixtures thereof, and the optional water soluble, pharmaceutically acceptable channeling agent is selected from the group consisting of sodium chloride, sodium phosphate, sucrose, lactose, dextrose, mannitol, xylitol, sorbitol, maltitol, sodium acetate, sodium citrate, hydroxypropyl cellulose, hydroxypropyl methylcellulose, hydroxyethyl cellulose, povidone, polyethylene oxide, polyethylene glycol, and mixtures thereof.

12. The pharmaceutical dosage form of claim 11 wherein the pharmaceutical dosage form provides a maximum blood plasma concentration (Cₘₐₓ) within the range of about 80% to about 125% of about 20 ng/mL of cyclobenzaprine HCl and an AUC₀₋ₜ within the range of about 80% to about 125% of about 740 nghr/mL following oral administration of a single 30 mg cyclobenzaprine HCl MR Capsule.

13. The pharmaceutical dosage form of claim 11 which exhibits a Cₘₐₓ below 8.0 ng/mL in vivo when co-administered to an adult as a capsule in a dosage form of 15 mg with eight ounces of 20 vol. % ethanol.

14. The pharmaceutical dosage form of claim 11 which exhibits a Cₘₐₓ below 10.0 ng/mL in vivo when co-administered to an adult as a capsule in a dosage form of 15 mg with eight ounces of 40 vol. % ethanol.

15. (canceled)

16. The pharmaceutical dosage form of claim 8, wherein the drug release profile substantially corresponds to the following pattern: after 2 hours, no more than about 40% of the total active is released; after 4 hours, from about 40-65% of the total active is released; after 8 hours, from about 60-85% of the total active is released; and after 12 hours, from about 75-85% of the total active is released.

17. The pharmaceutical dosage form of claim 8, wherein there is no water soluble, pharmaceutically acceptable channeling agent.

18. The pharmaceutical dosage form of claim 8, wherein the core particles are seal coated.

19. The pharmaceutical dosage form of claim 1, wherein:
the dosage form exhibits at least about 90% dissolution stability in capsule form in percentage dissolved in water, after a three month storage period under 40° C. and 75% relative humidity, relative to a baseline of 100% dissolution in water as measured at the onset of the storage period.

20. The dosage form of claim 19 wherein the dissolution stability is at least about 95%.
